(19) United States
(12) Patent Application Publication
(10) Pub. No.: US 2024/0392384 A1
(43) Pub. Date: Nov. 28, 2024

(54) DIAGNOSIS AND TREATMENT OF DISEASES AND CONDITIONS OF THE INTESTINAL TRACT

(71) Applicant: Flagship Pioneering Innovations VI, LLC, Cambridge, MA (US)

(72) Inventors: Douglas Gowers COLE, Wayland, MA (US); Jason PARK, West Newton, MA (US); Sabrina Yusang YANG, Cambridge, MA (US); Boris HAYETE, Needham, MA (US); Andrew John GASPARRINI, Boston, MA (US)

(21) Appl. No.: 18/694,571

(22) PCT Filed: Sep. 23, 2022
(86) PCT No.: PCT/US2022/076933

§ 371 Date: Mar. 22, 2024

Related U.S. Application Data
(60) Provisional application No. 63/247,450, filed on Sep. 23, 2021.

(51) Int. Cl. C12Q 1/6886; C12Q 1/689

(52) U.S. Cl. CPC C12Q 1/6886; C12Q 1/689

(57) ABSTRACT

The invention relates to diagnostic and therapeutic methods for colorectal cancer.

### SEQUENCE LISTING

The instant application contains a Sequence Listing which has been submitted electronically in XML format and is hereby incorporated by reference in its entirety. Said XML copy, created on Sep. 14, 2022, is named 51373-015WO1_Sequence_Listing_9_14_22.xml and is 4,610,094 bytes in size.

### FIELD OF THE INVENTION

The invention relates to diagnostic and therapeutic methods for colorectal cancer.

### BACKGROUND

Mammals are colonized by microorganisms in the gastrointestinal (GI) tract, on the skin, and in other epithelial and tissue niches. The gastrointestinal tract of a healthy individual harbors an abundant and diverse microbial community. It is a complex system, providing an environment or niche for a community of many different species or organisms, including diverse strains of bacteria. Hundreds of different species may form a commensal community in the GI tract in a healthy person, and this complement of organisms evolves from the time of birth and is believed to form a functionally mature microbial population by about 3 years of age. Interactions between microbial strains in these populations and between microorganisms and the host, e.g., interactions with the host's immune system, shape the community structure, with availability of and competition for resources affecting the distribution of microorganisms.

A healthy microbiome may provide a subject with multiple benefits, including colonization resistance to a broad spectrum of pathogens, essential nutrient biosynthesis and absorption, and immune stimulation that plays a role in maintaining a healthy gut epithelium and appropriately controlled systemic immunity. Conversely, an unhealthy (e.g., dysregulated) microbiome may be associated with a disease state.

There is a need for methods for addressing problems in healthcare through assessment of the microbiome.

### SUMMARY OF THE INVENTION

In one aspect, the disclosure features a method of diagnosing colorectal cancer (CRC) in a subject, the method comprising determining a level of one or more of SEQ ID NOs: 1-318 in a sample from the subject, wherein a level of one or more of SEQ ID NOs: 1-318 that is changed relative to a respective reference level for SEQ ID NOs: 1-318 indicates that the subject is likely to have a CRC.

In another aspect, the disclosure features a method for determining an increased risk of CRC in a subject, comprising the steps of (a) measuring the nucleic acid level of one or more of SEQ ID NOs: 1-318 in a sample collected from the subject using amplification and one or more pairs of primers specific for each of the one or more of SEQ ID NOs: 1-318; and (b) using the amplification results to determine whether the level of one or more of SEQ ID NOs: 1-318 is changed relative to a respective reference level for SEQ ID NOs: 1-318, thereby determining an increased risk of CRC for the subject.

In some embodiments, the method comprises determining or measuring a level of at least two, at least three, at least four, at least five, at least six, at least seven, at least eight, at least nine, at least ten, at least fifteen, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, or at least 310 of SEQ ID NOs: 1-318 in the sample from the subject.

In some embodiments, the method comprises determining or measuring a level of all 318 of SEQ ID NOs: 1-318 in the sample from the subject.

In some embodiments, a level of one or more of SEQ ID NOs: 1-318 is changed relative to the respective reference level for SEQ ID NO: 1-318 in the sample from the subject and the method further comprises administering an anti-CRC therapy to the subject.

In some embodiments, the anti-CRC therapy comprises one or more of a surgery, a chemotherapy, an immunotherapy targeting vascular endothelial growth factor (VEGF), an immunotherapy targeting epidermal growth factor receptor (EGFR), an anti-BRAF therapy, a kinase inhibitor, and a checkpoint inhibitor. In some embodiments, the chemotherapy is 5-fluorouracil (5-FU), capecitabine (XELODA®), irinotecan (CAMPTOSAR®), oxaliplatin (ELOXATIN®), or trifluridine and tipiracil (LONSURF®); the immunotherapy targeting VEGF is bevacizumab (AVASTIN®), ramucirumab (CYRAMZA®), or ziv-aflibercept (ZALTRAP®); the immunotherapy targeting EGFR is cetuximab (ERBITUX®) or panitumumab (VECTIBIX®); the anti-BRAF therapy is encorafenib (BRAFTOVI®); the kinase inhibitor is regorafenib (STIVARGA®); or the checkpoint inhibitor is pembrolizumab (KEYTRUDA®), nivolumab (OPDIVO®), or ipilimumab (YERVOY®).

In some embodiments, the anti-CRC therapy further comprises treatment by fecal microbiota transplant (FMT).

In some embodiments, the reference level is a pre-assigned level. In some embodiments, the reference level is a level in a set of samples from a reference population (e.g., a reference population of humans). In some embodiments, the reference population is a population of healthy subjects (e.g., healthy human subjects).

In some embodiments, the level of each of SEQ ID NOs: 1-318 that is determined or measured in the sample from the subject is a nucleic acid level. In some embodiments, the nucleic acid level is a DNA level.

In some embodiments, the change is a decrease relative to the reference level. In some embodiments, the levels of at least 10%, at least 20%, at least 30%, at least 40%, at least 50%, at least 60%, at least 70%, at least 80%, at least 90%, or more than 90% of the sequences for which a reference level is determined or measured are decreased relative to a respective reference level for the sequence.

In some embodiments, the change is an increase relative to the reference level. In some embodiments, the levels of at least 10%, at least 20%, at least 30%, at least 40%, at least 50%, at least 60%, at least 70%, at least 80%, at least 90%, or more than 90% of the sequences for which a reference level is determined or measured are increased relative to a respective reference level for the sequence.

In some embodiments, the sample comprises a sample of the microbiota of the subject. In some embodiments, the sample is a fecal sample.

In another aspect, the disclosure features a kit for diagnosing colorectal cancer (CRC) in a subject, the kit comprising (a) polypeptides or polynucleotides capable of determining the level of one or more of SEQ ID NOs: 1-318 in a sample from the subject; and optionally (b) instructions for use of the polypeptides or polynucleotides to determine the level of one or more of SEQ ID NOs: 1-318 in the sample from the subject, wherein a change in the level of one or more of SEQ ID NOs: 1-318 relative to a respective reference level for SEQ ID NOs: 1-318 indicates that the subject is likely to have a CRC.

In another aspect, the disclosure features a method of diagnosing CRC in a subject, the method comprising determining a level of each of SEQ ID NOs: 1-318 in a sample from the subject, wherein a level of at least 50% of SEQ ID NOs: 1-318 that is changed (e.g., increased or decreased) relative to a respective reference level for SEQ ID NOs: 1-318 indicates that the subject is likely to have a CRC.

In some embodiments, a level of at least 50% of SEQ ID NOs: 1-318 is changed (e.g., increased or decreased) relative to a respective reference level for SEQ ID NOs: 1-318 in the sample from the subject and the method further comprises administering an anti-CRC therapy to a subject.

In some embodiments, a level of at least 60%, 70%, 80%, 90%, or 95% of SEQ ID NOs: 1-318 that is changed (e.g., increased or decreased) in a sample from the subject relative to a respective reference level for SEQ ID NOs: 1-318 indicates that the subject is likely to have a CRC.

In another aspect, the disclosure features a method of treating a subject having a CRC by a method selected from the group consisting of surgery, chemotherapy, an immunotherapy targeting VEGF, an immunotherapy targeting EGFR, an anti-BRAF therapy, a kinase inhibitor, a checkpoint inhibitor, and FMT, wherein the subject was diagnosed as having a CRC by any of the methods provided herein.

In another aspect, the disclosure features a method of treating a subject having a CRC, the method comprising the steps of (a) measuring the nucleic acid level of one or more of SEQ ID NOs: 1-318 in a sample collected from the subject, wherein a level of one or more of SEQ ID NOs: 1-318 that is changed relative to a respective reference level for SEQ ID NOs: 1-318 indicates that the subject is likely to have a CRC; and (b) treating a subject who has been determined to have an increased risk of CRC with a method selected from the group consisting of surgery, chemotherapy, an immunotherapy targeting VEGF, an immunotherapy targeting EGFR, an anti-BRAF therapy, a kinase inhibitor, a checkpoint inhibitor, and FMT. In some embodiments, the chemotherapy is 5-fluorouracil (5-FU), capecitabine (XELODA®), irinotecan (CAMPTOSAR®), oxaliplatin (ELOXATIN®), or trifluridine and tipiracil (LONSURF®); the immunotherapy targeting VEGF is bevacizumab (AVASTIN®), ramucirumab (CYRAMZA®), or ziv-aflibercept (ZALTRAP®); the immunotherapy targeting EGFR is cetuximab (ERBITUX®) or panitumumab (VECTIBIX®); the anti-BRAF therapy is encorafenib (BRAFTOVI®); the kinase inhibitor is regorafenib (STIVARGA®); or the checkpoint inhibitor is pembrolizumab (KEYTRUDA®), nivolumab (OPDIVO®), or ipilimumab (YERVOY®).

In some embodiments, the nucleic acid level of the one or more of SEQ ID NOs: 1-318 in the sample collected from the subject are measured using amplification and one or more pairs of primers specific for each of the one or more of SEQ ID NOs: 1-318, and the amplification results are used to determine whether the level of one or more of SEQ ID NOs: 1-318 is changed relative to a respective reference level for SEQ ID NOs: 1-318.

In some embodiments, the method comprises measuring the nucleic acid level of at least two, at least three, at least four, at least five, at least six, at least seven, at least eight, at least nine, at least ten, at least fifteen, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, or at least 310 of SEQ ID NOs: 1-318 in the sample from the subject. In some embodiments, the method comprises measuring the nucleic acid level of all 318 of SEQ ID NOS: 1-318 in the sample from the subject.

In some embodiments, the reference level is a pre-assigned level. In some embodiments, the reference level is a level in a set of samples from a reference population (e.g., a reference population of humans). In some embodiments, the reference population is a population of healthy subjects (e.g., healthy human subjects).

In some embodiments, the nucleic acid level is a DNA level.

In some embodiments, the change is a decrease relative to the reference level. In some embodiments, the levels of at least 10%, at least 20%, at least 30%, at least 40%, at least 50%, at least 60%, at least 70%, at least 80%, at least 90%, or more than 90% of the sequences for which a level is measured are decreased relative to a respective reference level for the sequence.

In some embodiments, the change is an increase relative to the reference level. In some embodiments, the levels of at least 10%, at least 20%, at least 30%, at least 40%, at least 50%, at least 60%, at least 70%, at least 80%, at least 90%, or more than 90% of the sequences for which a level is measured are increased relative to a respective reference level for the sequence.

In some embodiments, the sample comprises a sample of the microbiota of the subject. In some embodiments, the sample is a fecal sample.

In another aspect, the disclosure features a method of treating a subject having a CRC, the method comprising the steps of (a) measuring the nucleic acid level of each of SEQ ID NOs: 1-318 in a sample collected from the subject, wherein a level of at least 50% of SEQ ID NOs: 1-318 that is changed relative to a respective reference level for SEQ ID NOs: 1-318 indicates that the subject is likely to have a CRC, thereby determining an increased risk of CRC for the subject; and (b) treating a subject who has been determined to have an increased risk of CRC with a method selected from the group consisting of surgery, chemotherapy, an immunotherapy targeting VEGF, an immunotherapy targeting EGFR, an anti-BRAF therapy, a kinase inhibitor, a checkpoint inhibitor, and FMT. Examples of the therapies are above.

In some aspects, the nucleic acid levels of each of SEQ ID NOs: 1-318 in the sample collected from the subject are measured using amplification and one or more pairs of primers specific for each of SEQ ID NOs: 1-318, and the amplification results are used to determine whether the level of each of SEQ ID NOs: 1-318 is changed relative to a respective reference level for SEQ ID NOs: 1-318.

### BRIEF DESCRIPTION OF THE DRAWINGS

FIG. 1 is a graph showing the out-of-cohort performance (as percent specificity and sensitivity) of metagenomic markers for distinguishing colorectal cancer (CRC) patients from control patients. AUC=area under curve. nBoot=100; nMetagenomic markers=589; w.a. AUROC=0.717; Wilcox p-value=1.82×10⁻¹¹.

FIG. 2A is a graph showing the score and rank of a model comprising 318 metagenomic markers and a null model.

FIG. 2B is a heat map showing the level (scaled reads per kilobase of transcript per million mapped reads (RPKM)) of 318 metagenomic markers in CRC and control (CTR) patients.

### DETAILED DESCRIPTION OF THE INVENTION

### Definitions

The term “changed.” as used herein, refers to an observable difference in the level of a marker in a subject (e.g., in a sample from the subject), as determined using techniques and methods known in the art for the measurement of the marker. A marker level that is changed in a subject may result in a difference of at least 1% (e.g., at least 5%, 10%, 15%, 20%, 25%, 30%, 35%, 40%, 45%, 50%, 55%, 60%, 65%, 70%, 75%, 80%, 85%, 90%, 95%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) more or less than a reference level (e.g., a level from a healthy subject or a level prior to treatment) (e.g., up to 100% or up to 100-fold relative to the reference level). In some embodiments, the change is an increase in the level of a marker in a subject. Increasing the marker level in a subject may result in an increase of at least 1% (e.g., at least 5%, 10%, 15%, 20%, 25%, 30%, 35%, 40%, 45%, 50%, 55%, 60%, 65%, 70%, 75%, 80%, 85%, 90%, 95%, 98%, 99%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the reference level (e.g., up to 100% or up to 100-fold relative to the reference level). In other embodiments, the change is a decrease the level of a marker in a subject. Decreasing the marker level in a subject may result in a decrease of at least 1% (e.g., at least 5%, 10%, 15%, 20%, 25%, 30%, 35%, 40%, 45%, 50%, 55%, 60%, 65%, 70%, 75%, 80%, 85%, 90%, 95%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the reference level (e.g., up to 100% or up to 100-fold relative to the reference level).

In some embodiments, the change in the level of a portion of the markers analyzed is an increase, while the change in the level of another portion of the markers analyzed is a decrease. In some embodiments, the change in at least 10%, 20%, 30%, 40%, 50%, 60%, 70%, 80%, 90%, 95%, or more (e.g., 100%) of the markers analyzed is an increase relative to a reference level. In some embodiments, the change in at least 10%, 20%, 30%, 40%, 50%, 60%, 70%, 80%, 90%, 95%, or more (e.g., 100%) of the markers analyzed is a decrease relative to a reference level.

The term “pharmaceutical composition,” as used herein, represents a composition formulated with a pharmaceutically acceptable excipient. For example, a “pharmaceutical composition” can be a composition that is manufactured or sold with the approval of a governmental regulatory agency as part of a therapeutic regimen for the treatment of a disease, disorder, or condition in a mammal, intended for such use, or in development for such use. In some examples, the pharmaceutical composition is a pre-approved composition.

The term “subject.” as used herein, represents a human or non-human animal (e.g., a mammal).

“Treatment” and “treating.” as used herein, refer to the medical management of a subject with the intent to improve, ameliorate, stabilize, prevent, or cure a disease, disorder, or condition. This term includes active treatment (treatment directed to improve the disease, disorder, or condition); causal treatment (treatment directed to the cause of the associated disease, disorder, or condition); palliative treatment (treatment designed for the relief of symptoms of the disease, disorder, or condition); preventative treatment (treatment directed to minimizing or partially or completely inhibiting the development of the associated disease, disorder, or condition); and supportive treatment (treatment employed to supplement another therapy).

The term “cancer,” as used herein, refers to a disease caused by an uncontrolled division of abnormal cells in a part of the body. In one instance, the cancer is a colorectal cancer (CRC). CRC includes cancers of the colon and/or rectum, e.g., adenocarcinoma of the colon or rectum. The cancer (e.g., CRC) may be locally advanced or metastatic, e.g., may be a Stage I, Stage II, Stage III, or Stage IV cancer.

### I. METHODS FOR DIAGNOSING AND TREATING COLORECTAL CANCER

The invention is based, in part, on the discovery that levels of gut microbiome biomarkers (i.e., markers of bacterial origin), which may be referred to as co-evolved molecules, can be used to identify patients having a colorectal cancer (CRC). Accordingly, the disclosure provides methods of diagnosing, treating, and monitoring subjects (e.g., human patients) based on this discovery.

### Diagnostic Methods and Methods of Treatment

In some aspects, the disclosure features methods of diagnosing colorectal cancer (CRC) in a subject (e.g., a human subject), the methods comprising determining a level of one or more of SEQ ID NOs: 1-318 in a sample from the subject, wherein a level of one or more of SEQ ID NOs: 1-318 that is changed relative to a respective reference level for SEQ ID NOs: 1-318 indicates that the subject is likely to have a CRC. SEQ ID NOs: 1-318 are bacterial sequences.

In some aspects, the disclosure features a method for determining an increased risk of CRC in a subject, comprising the steps of (a) measuring the nucleic acid level of one or more of SEQ ID NOs: 1-318 in a sample collected from the subject using amplification and one or more pairs of primers specific for each of the one or more of SEQ ID NOs: 1-318; and (b) using the amplification results to determine whether the level of one or more of SEQ ID NOs: 1-318 is changed relative to a respective reference level for SEQ ID NOs: 1-318, thereby determining an increased risk of CRC for the subject.

In some aspects, the disclosure features a method of treating a subject having a CRC, the method comprising the steps of (a) measuring the nucleic acid level of one or more of SEQ ID NOs: 1-318 in a sample collected from the subject, wherein a level of one or more of SEQ ID NOs: 1-318 that is changed relative to a respective reference level for SEQ ID NOs: 1-318 indicates that the subject is likely to have a CRC; and (b) treating a subject who has been determined to have an increased risk of CRC with a method selected from the group consisting of surgery, chemotherapy, an immunotherapy targeting VEGF, an immunotherapy targeting EGFR, an anti-BRAF therapy, a kinase inhibitor, a checkpoint inhibitor, and FMT. In some embodiments, the nucleic acid level of the one or more of SEQ ID NOs: 1-318 in the sample collected from the subject are measured using amplification and one or more pairs of primers specific for each of the one or more of SEQ ID NOs: 1-318, and the amplification results are used to determine whether the level of one or more of SEQ ID NOs: 1-318 is changed relative to a respective reference level for SEQ ID NOs: 1-318.

In some aspects, the disclosure features a method of treating a subject having a CRC, the method comprising the steps of (a) measuring the nucleic acid level of each of SEQ ID NOS: 1-318 in a sample collected from the subject, wherein a level of at least 50% of SEQ ID NOs: 1-318 that is changed relative to a respective reference level for SEQ ID NOs: 1-318 indicates that the subject is likely to have a CRC, thereby determining an increased risk of CRC for the subject; and (b) treating a subject who has been determined to have an increased risk of CRC with a method selected from the group consisting of surgery, chemotherapy, an immunotherapy targeting VEGF, an immunotherapy targeting EGFR, an anti-BRAF therapy, a kinase inhibitor, a checkpoint inhibitor, and FMT. In some embodiments, the nucleic acid levels of each of SEQ ID NOs: 1-318 in the sample collected from the subject are measured using amplification and one or more pairs of primers specific for each of SEQ ID NOs: 1-318, and the amplification results are used to determine whether the level of each of SEQ ID NOs: 1-318 is changed relative to a respective reference level for SEQ ID NOs: 1-318.

In some embodiments, the sample comprises a sample of the microbiota (e.g., the gut microbiota) of the subject. In some embodiments, the sample is a fecal sample. In some embodiments, the sample is a colon or rectal biopsy.

In some embodiments, the method comprises determining or measuring a level of at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, or at least 315 of SEQ ID NOs: 1-318 in a sample from the subject, e.g., comprises determining or measuring a level of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 215-220, 220-225, 225-230, 230-235, 235-240, 240-245, 245-250, 250-255, 255-260, 260-265, 265-270, 270-275, 275-280, 280-285, 285-290, 290-295, 295-300, 300-305, 305-310, 310-315, or 315-318 of SEQ ID NOs: 1-318 in the sample from the subject. In some embodiments, the method comprises determining or measuring a level of all 318 of SEQ ID NOs: 1-318 in a sample from the subject.

In some embodiments, a change in the level of at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, or at least 315 of SEQ ID NOs: 1-318 in a sample from the subject, e.g., a change in the level of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 215-220, 220-225, 225-230, 230-235, 235-240, 240-245, 245-250, 250-255, 255-260, 260-265, 265-270, 270-275, 275-280, 280-285, 285-290, 290-295, 295-300, 300-305, 305-310, 310-315, or 315-318 of SEQ ID NOs: 1-318 in the sample from the subject relative to a respective reference level for SEQ ID NOs: 1-318 indicates that the subject is likely to have a CRC. In some embodiments, a change in the level of all 318 of SEQ ID NOs: 1-318 relative to a respective reference level for SEQ ID NOs: 1-318 indicates that the subject is likely to have a CRC.

In some embodiments, a change in the level of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, at least 98%, at least 99%, or 100% of the total number of SEQ ID NOs: 1-318 measured in a sample from the subject relative to a respective reference level for SEQ ID NOs: 1-318 indicates that the subject is likely to have a CRC.

In some embodiments, a threshold number of one or more of SEQ ID NOs: 1-318 is changed relative to the respective reference level for SEQ ID NO: 1-318 in the sample from the subject (e.g., a number of SEQ ID NOs: 1-318 that has been determined to indicate that the patient is likely to have a CRC, e.g., a level of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, or at least 315 of SEQ ID NOs: 1-318 in a sample from the subject (e.g., a level of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 215-220, 220-225, 225-230, 230-235, 235-240, 240-245, 245-250, 250-255, 255-260, 260-265, 265-270, 270-275, 275-280, 280-285, 285-290, 290-295, 295-300, 300-305, 305-310, 310-315, or 315-318)) of SEQ ID NOs: 1-318 is changed relative to the respective reference level for SEQ ID NO: 1-318 in the sample from the subject and the method further comprises administering an anti-CRC therapy to a subject.

The anti-CRC therapy for any method herein may be any medicament, treatment, or combination thereof suitable for the treatment of CRC. In some aspects, the anti-CRC therapy comprises one or more of a surgery; a chemotherapy (e.g., a chemotherapy comprising 5-fluorouracil (5-FU), capecitabine (XELODA®,); irinotecan (CAMPTOSAR®); oxaliplatin (ELOXATIN®); trifluridine and tipiracil (LONSURF®); or 5-fluorouracil (FU), leucovorin (LV), and either oxaliplatin (FOLFOX) or irinotecan (FOLFIRI)); an anti-vascular endothelial growth factor (VEGF) therapy, e.g., an immunotherapy targeting VEGF (e.g., bevacizumab (AVASTIN®), ramucirumab (CYRAMZA®), or ziv-aflibercept (ZALTRAP®)); an immunotherapy targeting epidermal growth factor receptor (EGFR) (e.g., cetuximab (ERBITUX®) or panitumumab (VECTIBIX®)); an anti-BRAF therapy (e.g., encorafenib (BRAFTOVI®)); a kinase inhibitor (e.g., regorafenib (STIVARGA®)); and a checkpoint inhibitor (e.g., pembrolizumab (KEYTRUDA®), nivolumab (OPDIVO®), or ipilimumab (YERVOY®)). In some aspects, the anti-CRC therapy further comprises treatment by fecal microbiota transplant (FMT).

In some embodiments, the respective reference level for SEQ ID NOs: 1-318 is a pre-assigned level of one of SEQ ID NOs: 1-318.

In some embodiments, the respective reference level for SEQ ID NOs: 1-318 is a level in a set of samples from a reference population, e.g., a population of healthy subjects (e.g., a population of subjects not having a CRC and/or a population of subjects having a healthy gut microbiome). In some embodiments, the healthy subjects are healthy human subjects.

In some embodiments, the level of each of SEQ ID NOs: 1-318 that is determined in a sample from the subject is a nucleic acid level, e.g., a DNA level or an RNA level. In some embodiments, the nucleic acid level is a DNA level, which may be detected, e.g., using a PCR-based method. In other embodiments, detection of a level of one or more of SEQ ID NOs: 1-318 can optionally comprise, for example, detection of RNA levels, which can be achieved by, e.g., RT-PCR, RNA-Seq, and/or methods including the use of microarrays, as are known in the art. In other embodiments, the methods can focus on the detection of protein levels, which can be carried out using standard approaches (e.g., immunoassay-based approaches).

In some embodiments, the change in a level of one or more of SEQ ID NOs: 1-318 in the sample from the subject is a decrease relative to the respective reference level for SEQ ID NOs: 1-318 (e.g., a decrease of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, e.g., a decrease of, e.g., at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the respective reference level for SEQ ID NOs: 1-318; or a decrease of 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, or 95-100% relative to the respective reference level for SEQ ID NOs: 1-318).

In some aspects, the levels of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of the sequences for which a level is determined or measured in the sample from the subject (i.e., one or more of SEQ ID NOs: 1-318) are decreased relative to a respective reference level for the sequence, e.g., 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, or 95-100% of the sequences for which a level is determined or measured are decreased relative to a respective reference level for the sequence.

In some embodiments, the change in a level of one or more of SEQ ID NOs: 1-318 in the sample from the subject is an increase relative to the respective reference level for SEQ ID NOs: 1-318 (e.g., an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more than 100%, e.g., an increase of, e.g., 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold, relative to the respective level for SEQ ID NOs: 1-318; or an increase of 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, 95-100%, or more than 100%, relative to the respective reference level for SEQ ID NOs: 1-318).

In some aspects, the levels of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of the sequences for which a level is determined or measured in the sample from the subject (i.e., one or more of SEQ ID NOs: 1-318) are increased relative to a respective reference level for the sequence, e.g., 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, or 95-100% of the sequences for which a level is determined or measured are increased relative to a respective reference level for the sequence.

In some aspects, a level of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of SEQ ID NOs: 1-318 that is increased in a sample from the subject relative to a respective reference level for SEQ ID NOs: 1-318 indicates that the subject is likely to have a CRC.

Determination of whether a difference detected is significant can be carried out using standard methods, as well as statistical analysis. In some embodiments, a difference detected is a change of at least 5%, 10%, 20%, 30%, 40%, 50%, 75%, 100%, or more, e.g., at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold, relative to a reference level.

### II. ARTICLES OF MANUFACTURE AND KITS

In another aspect of the invention, an article of manufacture or kit containing materials useful for the diagnosis, prognostic assessment, and/or treatment of individuals is provided.

In one aspect, the disclosure features a kit or article of manufacture for diagnosing colorectal cancer (CRC) in a subject, the kit comprising (a) reagents for determining a level of one or more of SEQ ID NOs: 1-318 in a sample from the subject (e.g., polynucleotides or polypeptides capable of use in determining the level of one or more of SEQ ID NOs: 1-318 in a sample from the subject); and optionally (b) instructions for use of the polynucleotides or polypeptides to determine the level of one or more of SEQ ID NOs: 1-318 in the sample from the subject, wherein a change in the level of one or more of SEQ ID NOs: 1-318 relative to a respective reference level for SEQ ID NOs: 1-318, as described herein, indicates that the subject is likely to have a CRC.

In some aspects, the reagents for determining a level of one or more of SEQ ID NOS: 1-318 in a sample from the subject comprise one or more polynucleotides (e.g., PCR primers) that hybridize to a complement of a locus of one or more of SEQ ID NOs: 1-318 under stringent conditions and may be used to amplify all or a portion of any one or more of SEQ ID NOs: 1-318, as described herein. In some aspects, the instructions indicate that the one or more oligonucleotides (e.g., PCR primers) may be used to evaluate the presence and/or level of one or more of SEQ ID NOs: 1-318 in a sample from the subject and provide instructions for using the polynucleotide(s) for evaluating the presence and/or level of one or more of SEQ ID NOs: 1-318 in the sample. In some embodiments, reagents are included for determining a level of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of the sequences of SEQ ID NOs: 1-318.

For polynucleotide-based articles of manufacture or kits, the article of manufacture or kit may include, for example: (1) an oligonucleotide, e.g., a detectably labeled oligonucleotide, which hybridizes to a nucleic acid sequence encoding a protein, (2) a pair of primers useful for amplifying a nucleic acid molecule, or (3) a microarray comprising multiple oligonucleotide probes. For protein-based articles of manufacture or kits, the article of manufacture or kit may include, for example, one or more antibody-based reagents. The article of manufacture or kit can also include, e.g., a buffering agent, a preservative, or a protein-stabilizing agent. The article of manufacture or kit can further include components necessary for detecting the detectable label (e.g., an enzyme or a substrate). The article of manufacture or kit can further include components necessary for analyzing the sequence of a sample (e.g., a restriction enzyme or a buffer). The article of manufacture or kit can also contain a control sample or a series of control samples that can be assayed and compared to the test sample (e.g., a reference sample, as described herein). Each component of the article of manufacture or kit can be enclosed within an individual container and all of the various containers can be within a single package, along with instructions for interpreting the results of the assays performed using the kit.

The following examples are meant to illustrate the invention. They are not meant to limit the invention in any way.

### III. EXAMPLES

### Example 1. Identification of Markers

### A. Assembly and Annotation of Metagenomic Markers

Paired end reads from healthy Human Microbiome Project 1 (HMP1; Human Microbiome Project Consortium, Nature, 486 (7402): 207-214, 2012) individuals were downloaded from the National Center for Bioinformatics (NCBI) Short Read Archive (SRA) and assembled using metaSPAdes (cab.spbu.ru/software/spades/). Metagenomic markers were annotated using antiSMASH4.0 (docs.antismash.secondarymetabolites.org/) with the following non-default parameters:-c 3--smcogs--disable-embl. Annotated metagenomic markers were clustered using all vs. all diamond (https://github.com/bbuchfink/diamond) blastx. Blastx results were filtered using a python script requiring (a) E-value <1×10⁻⁵. (b) 90% coverage of length of coding sequence, and (c)>50% of coding sequences in a metagenomic marker present. Metagenomic markers were then grouped using markov clustering, resulting in a dereplicated library of 8,211 representative metagenomic markers identified in healthy human gut metagenomes.

### B. EMP Selection

A subset of metagenomic markers prevalent in healthy cohorts, referred to as essential microbial products (EMPs), were identified by clustering metagenomic markerpresence/absence across 592 healthy patients from various geographic, genetic, and lifestyle backgrounds. Clusters with a mean prevalence >0.7 and z-score >10 within cohort were selected. To take sample imbalance into account, a proportion test was performed to assess stability across cohorts. This resulted in 1321 EMPs. For diagnostic analyses, metagenomic marker annotations were subsetted from the full dataset, resulting in 1171 EMPs. Metagenomic markers annotations were further subsetted, resulting in 590 EMPs.

### C. Quantification of Metagenomic Markers Across Cohorts

To quantify metagenomic marker abundance across metagenomes, raw forward reads were mapped to the library of 8,211 metagenomic markers using diamond as described above. Normalized abundance of metagenomic markers was calculated such that abundance equals number of reads mapping divided by cumulative length of coding sequences in kilobase pairs divided by number of raw reads mapped.

### D. CRC Data

Raw metagenomic reads from papers analyzed in the Wirbel et al., Nat Med, 25(4): 679-689, 2019 meta-analysis were downloaded from NCBI. After removing studies that contained only 16S sequencing, 575 samples (285 colorectal cancer (CRC) and 290 control) remained. The CRC population included patients with all stages of CRC (Stage 0:10 patients; Stage I: 65 patients; Stage II: 70 patients; the median stage was stage II.

### E. Data Preprocessing

EMP abundance data were transformed using hyperbolic arcsine, corrected for batch effects using ComBat (rdrr.io/bioc/sva/man/ComBat.html) from the sva package in R, and centered and scaled.

### F. Modeling

For testing predictive performance, logistic regression models of the form diagnosis˜metagenomic markers were fit with BootGlmV2 with nBoot=100 using leave-one-study-out cross validation. BootGlmV2 is an ensemble approach to regularized logistic regression using interactions. Out-of-cohort area under the receiver operating characteristic (ROC) curve was computed using the pROC package in R (cran.r-project.org/web/packages/pROC/PROC.pdf; Robin et al., BMC Bioinformatics, 12:77, 2011), with per-study AUC values weighted by study size. p-value on the ROC curve was computed using the Wilcoxon rank sum test (blog.revolutionanalytics.com/2017/03/auc-meets-u-stat.html). For feature selection, full models including all samples were fit with (a) actual data and, (b) data with the response variable randomly permuted using BootGlmV2 parameter randomize=TRUE. Features were ranked and considered significant with a score greater than the maximum score observed in the null (randomly permuted) model.

### G. Visualization

Heatmaps were constructed using pheatmap with binarized data and clustering of columns according to Ward's distance.

### H. Conclusions

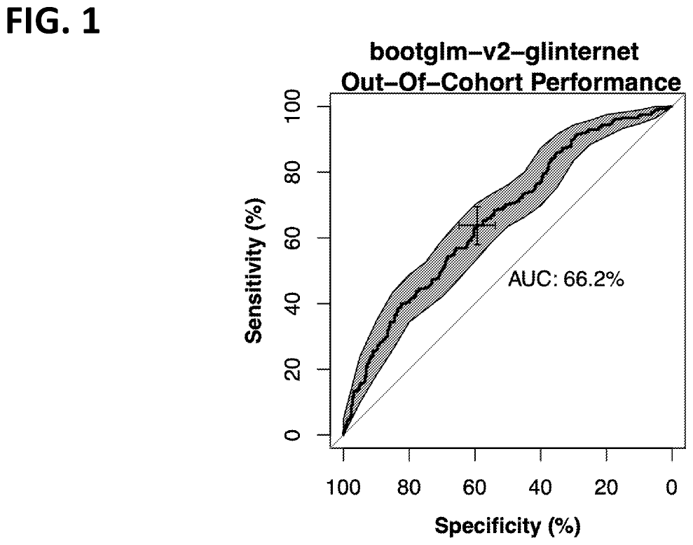

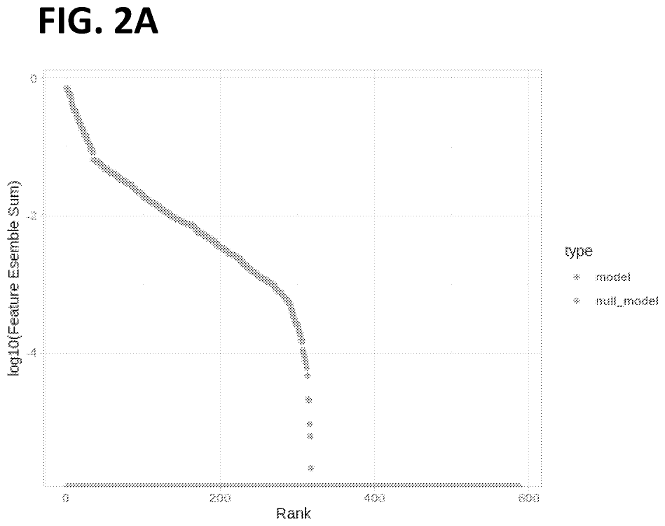

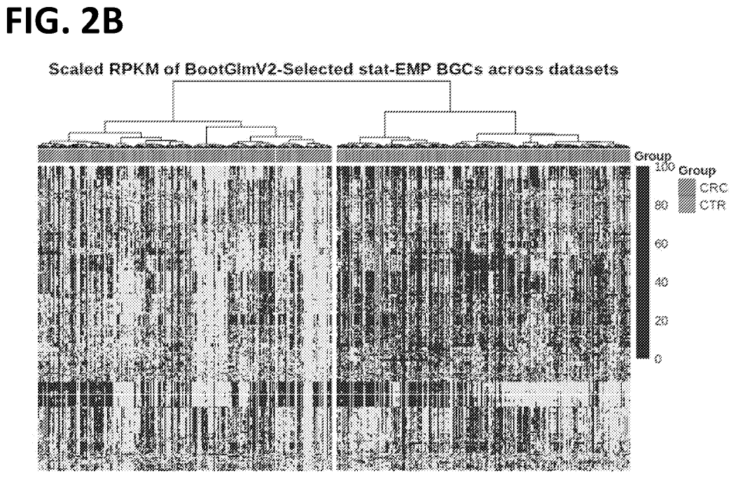

A set of 318 metagenomic markers (SEQ ID NOs: 1-318) were found to be significant predictors in a model discriminating between colorectal cancer (CRC) patients and control individuals (FIGS. 1, 2A, and 2B).

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

What is claimed is:

1. A method of diagnosing colorectal cancer (CRC) in a subject, the method comprising determining a level of one or more of SEQ ID NOs: 1-318 in a sample from the subject, wherein a level of one or more of SEQ ID NOs: 1-318 that is changed relative to a respective reference level for SEQ ID NOs: 1-318 indicates that the subject is likely to have a CRC.

2. A method for determining an increased risk of CRC in a subject, comprising the steps of:
(a) measuring the nucleic acid level of one or more of SEQ ID NOs: 1-318 in a sample collected from the subject using amplification and one or more pairs of primers specific for each of the one or more of SEQ ID NOs: 1-318; and
(b) using the amplification results to determine whether the level of one or more of SEQ ID NOs: 1-318 is changed relative to a respective reference level for SEQ ID NOs: 1-318, thereby determining an increased risk of CRC for the subject.

3. The method of claim 1 or 2, wherein the method comprises determining or measuring a level of at least two, at least three, at least four, at least five, at least six, at least seven, at least eight, at least nine, at least ten, at least fifteen, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, or at least 310 of SEQ ID NOs: 1-318 in the sample from the subject.

4. The method of claim 3, wherein the method comprises determining or measuring a level of all 318 of SEQ ID NOs: 1-318 in the sample from the subject.

5. The method of any one of claims 1-4, wherein a level of one or more of SEQ ID NOs: 1-318 is changed relative to the respective reference level for SEQ ID NO: 1-318 in the sample from the subject and the method further comprises administering an anti-CRC therapy to the subject.

6. The method of claim 5, wherein the anti-CRC therapy comprises one or more of a surgery, a chemotherapy, an immunotherapy targeting vascular endothelial growth factor (VEGF), an immunotherapy targeting epidermal growth factor receptor (EGFR), an anti-BRAF therapy, a kinase inhibitor, and a checkpoint inhibitor.

7. The method of claim 6, wherein the chemotherapy is 5-fluorouracil (5-FU), capecitabine (XELODA®), irinotecan (CAMPTOSAR®), oxaliplatin (ELOXATIN®), or trifluridine and tipiracil (LONSURF®); the immunotherapy targeting VEGF is bevacizumab (AVASTIN®), ramucirumab (CYRAMZA®), or ziv-aflibercept (ZALTRAP®); the immunotherapy targeting EGFR is cetuximab (ERBITUX®) or panitumumab (VECTIBIX®); the anti-BRAF therapy is encorafenib (BRAFTOVI®); the kinase inhibitor is regorafenib (STIVARGA®); or the checkpoint inhibitor is pembrolizumab (KEYTRUDA®), nivolumab (OPDIVO®), or ipilimumab (YERVOY®).

8. The method of any one of claims 5-7, wherein the anti-CRC therapy further comprises treatment by fecal microbiota transplant (FMT).

9. The method of any one of claims 1-8, wherein the reference level is a pre-assigned level.

10. The method of any one of claims 1-9, wherein the reference level is a level in a set of samples from a reference population.

11. The method of claim 10, wherein the reference population is a population of healthy subjects.

12. The method of any one of claims 1-11, wherein the level of each of SEQ ID NOs: 1-318 that is determined or measured in the sample from the subject is a nucleic acid level.

13. The method of claim 12, wherein the nucleic acid level is a DNA level.

14. The method of any one of claims 1-13, wherein the change is a decrease relative to the reference level.

15. The method of claim 14, wherein the levels of at least 10%, at least 20%, at least 30%, at least 40%, at least 50%, at least 60%, at least 70%, at least 80%, at least 90%, or more than 90% of the sequences for which a level is determined or measured are decreased relative to a respective reference level for the sequence.

16. The method of any one of claims 1-13, wherein the change is an increase relative to the reference level.

17. The method of claim 16, wherein the levels of at least 10%, at least 20%, at least 30%, at least 40%, at least 50%, at least 60%, at least 70%, at least 80%, at least 90%, or more than 90% of the sequences for which a level is determined or measured are increased relative to a respective reference level for the sequence.

18. The method of any one of claims 1-17, wherein the sample comprises a sample of the microbiota of the subject.

19. The method of any one of claims 1-18, wherein the sample is a fecal sample.

20. A kit for diagnosing colorectal cancer (CRC) in a subject, the kit comprising:
(a) polypeptides or polynucleotides capable of determining the level of one or more of SEQ ID NOs: 1-318 in a sample from the subject; and
(b) instructions for use of the polypeptides or polynucleotides to determine the level of one or more of SEQ ID NOs: 1-318 in the sample from the subject, wherein a change in the level of one or more of SEQ ID NOs: 1-318 relative to a respective reference level for SEQ ID NOs: 1-318 indicates that the subject is likely to have a CRC.

21. The kit of claim 20, wherein the kit comprises polypeptides or polynucleotides capable of determining the level of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of SEQ ID NOs: 1-318 in a sample from the subject.

22. A method of diagnosing CRC in a subject, the method comprising determining a level of each of SEQ ID NOs: 1-318 in a sample from the subject, wherein a level of at least 50% of SEQ ID NOs: 1-318 that is changed relative to a respective reference level for SEQ ID NOs: 1-318 indicates that the subject is likely to have a CRC.

23. The method of claim 22, wherein a level of at least 50% of SEQ ID NOs: 1-318 is changed relative to a respective reference level for SEQ ID NOs: 1-318 in the sample from the subject and the method further comprises administering an anti-CRC therapy to a subject.

24. The method of claim 22 or 23, wherein a level of at least 60%, 70%, 80%, 90%, or 95% of SEQ ID NOs: 1-318 that is changed in a sample from the subject relative to a respective reference level for SEQ ID NOs: 1-318 indicates that the subject is likely to have a CRC.

25. A method of treating a subject having a CRC by a method selected from the group consisting of—surgery, chemotherapy, an immunotherapy targeting VEGF, an immunotherapy targeting EGFR, an anti-BRAF therapy, a kinase inhibitor, a checkpoint inhibitor, and FMT, wherein the subject was diagnosed as having a CRC by a method of any one of claims 1-19 and 22-24.

26. A method of treating a subject having a CRC, the method comprising the steps of:
(a) measuring the nucleic acid level of one or more of SEQ ID NOs: 1-318 in a sample collected from the subject, wherein a level of one or more of SEQ ID NOs: 1-318 that is changed relative to a respective reference level for SEQ ID NOs: 1-318 indicates that the subject is likely to have a CRC; and
(b) treating a subject who has been determined to have an increased risk of CRC with a method selected from the group consisting of surgery, chemotherapy, an immunotherapy targeting VEGF, an immunotherapy targeting EGFR, an anti-BRAF therapy, a kinase inhibitor, a checkpoint inhibitor, and FMT.

27. The method of claim 25 or 26, wherein the chemotherapy is 5-fluorouracil (5-FU), capecitabine (XELODA®), irinotecan (CAMPTOSAR®), oxaliplatin (ELOXATIN®), or trifluridine and tipiracil (LONSURF®); the immunotherapy targeting VEGF is bevacizumab (AVASTIN®), ramucirumab (CYRAMZA®), or ziv-aflibercept (ZALTRAP®); the immunotherapy targeting EGFR is cetuximab (ERBITUX®) or panitumumab (VECTIBIX®); the anti-BRAF therapy is encorafenib (BRAFTOVI®); the kinase inhibitor is regorafenib (STIVARGA®); or the checkpoint inhibitor is pembrolizumab (KEYTRUDA®), nivolumab (OPDIVO®), or ipilimumab (YERVOY®).

28. The method of claim 26 or 27, wherein the nucleic acid level of the one or more of SEQ ID NOs: 1-318 in the sample collected from the subject are measured using amplification and one or more pairs of primers specific for each of the one or more of SEQ ID NOs: 1-318, and the amplification results are used to determine whether the level of one or more of SEQ ID NOs: 1-318 is changed relative to a respective reference level for SEQ ID NOs: 1-318.

29. The method of any one of claims 26-28, wherein the method comprises measuring the nucleic acid level of at least two, at least three, at least four, at least five, at least six, at least seven, at least eight, at least nine, at least ten, at least fifteen, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, or at least 310 of SEQ ID NOs: 1-318 in the sample from the subject.

30. The method of claim 29, wherein the method comprises measuring the nucleic acid level of all 318 of SEQ ID NOs: 1-318 in the sample from the subject.

31. The method of any one of claims 26-30, wherein the reference level is a pre-assigned level.

32. The method of any one of claims 26-31, wherein the reference level is a level in a set of samples from a reference population.

33. The method of claim 32, wherein the reference population is a population of healthy subjects.

34. The method of any one of claims 26-33, wherein the nucleic acid level is a DNA level.

35. The method of any one of claims 26-34, wherein the change is a decrease relative to the reference level.

36. The method of claim 35, wherein the levels of at least 10%, at least 20%, at least 30%, at least 40%, at least 50%, at least 60%, at least 70%, at least 80%, at least 90%, or more than 90% of the sequences for which a level is measured are decreased relative to a respective reference level for the sequence.

37. The method of any one of claims 26-34, wherein the change is an increase relative to the reference level.

38. The method of claim 37, wherein the levels of at least 10%, at least 20%, at least 30%, at least 40%, at least 50%, at least 60%, at least 70%, at least 80%, at least 90%, or more than 90% of the sequences for which a level is measured are increased relative to a respective reference level for the sequence.

39. The method of any one of claims 26-38, wherein the sample comprises a sample of the microbiota of the subject.

40. The method of any one of claims 26-39, wherein the sample is a fecal sample.

41. A method of treating a subject having a CRC, the method comprising the steps of:
(a) measuring the nucleic acid level of each of SEQ ID NOs: 1-318 in a sample collected from the subject, wherein a level of at least 50% of SEQ ID NOs: 1-318 that is changed relative to a respective reference level for SEQ ID NOs: 1-318 indicates that the subject is likely to have a CRC, thereby determining an increased risk of CRC for the subject; and
(b) treating a subject who has been determined to have an increased risk of CRC with a method selected from the group consisting of surgery, chemotherapy, an immunotherapy targeting VEGF, an immunotherapy targeting EGFR, an anti-BRAF therapy, a kinase inhibitor, a checkpoint inhibitor, and FMT.

42. The method of claim 41, wherein the nucleic acid levels of each of SEQ ID NOs: 1-318 in the sample collected from the subject are measured using amplification and one or more pairs of primers specific for each of SEQ ID NOs: 1-318, and the amplification results are used to determine whether the level of each of SEQ ID NOs: 1-318 is changed relative to a respective reference level for SEQ ID NOs: 1-318.

43. The method of claim 41 or 42, wherein the chemotherapy is 5-fluorouracil (5-FU), capecitabine (XELODA®), irinotecan (CAMPTOSAR®), oxaliplatin (ELOXATIN®), or trifluridine and tipiracil (LONSURF®); the immunotherapy targeting VEGF is bevacizumab (AVASTIN®), ramucirumab (CYRAMZA®), or ziv-aflibercept (ZALTRAP®); the immunotherapy targeting EGFR is cetuximab (ERBITUX®) or panitumumab (VECTIBIX®); the anti-BRAF therapy is encorafenib (BRAFTOVI®); the kinase inhibitor is regorafenib (STIVARGA®); or the checkpoint inhibitor is pembrolizumab (KEYTRUDA®), nivolumab (OPDIVO®), or ipilimumab (YERVOY®).
